(19) United States
(12) Patent Application Publication
(10) Pub. No.: US 2010/0112086 A1
(43) Pub. Date: May 6, 2010

(54) ELECTROLYTICALLY REDUCED WATER, HOT WATER FOR BATHING, AND METHOD FOR SUPPRESSION OF LUMPY FAT

(75) Inventors: Nobuhiko Miwa, Hiroshima (JP); Hiroshi Tamura, Hiroshima (JP); Keisou Iwai, Hiroshima (JP); Osamu Mishima, Hiroshima (JP); Suehiro Sakai, Hiroshima (JP); Katsunori Yuuki, Hiroshima (JP); Masanori Akioka, Hiroshima (JP)

Correspondence Address: YOUNG & THOMPSON, 209 Madison Street, Suite 500, Alexandria, VA, 22314, US

(73) Assignees: PREFECTURAL UNIVERSITY OF HIROSHIMA, Hiroshima-shi (JP); THE CHUGOKU ELECTRIC MANUFACTURING COMPANY, INCORPORATED, Hiroshima-shi (JP)

(21) Appl. No.: 12/529,895

(22) PCT Filed: Jul. 3, 2008
(86) PCT No.: PCT/JP2008/062043

§ 371 Date: Sep. 3, 2009

(30) Foreign Application Priority Data
Jul. 10, 2007 (JP) 2007-181222
Jun. 2, 2008 (JP) 2008-145078

(51) Int. Cl. A61K 33/00; C01B 5/00; C02F 1/461; A61P 43/00; A61Q 19/00

(52) U.S. Cl. 424/600; 423/580.1

(57) ABSTRACT

An electrolytically reduced water which consists of an alkali ion water prepared by ion exchange and has a dissolved hydrogen concentration of 0.20 ppm or above, an oxidation-reduction potential of −150 mV to −500 mV, and a hydrogen ion component of pH6.5-10.5 and which is quantitatively specified in the reducing power capable of inhibiting the oxidative deterioration of fat in the cutis through the elimination of active oxygen due to the characteristics of hydrogen, the oxidation-reduction potential characteristics of high reducing action, and alkaline characteristics and can activate the skin to inhibit the oxidative deterioration and suppress lumpy fat (cellulite).

### TECHNICAL FIELD

This invention relates to electrolytically reduced water obtained by ionizing tap water supplied, from waterworks.

The present invention also relates to hot water for bathing, which uses electrolytically reduced water obtained by ionizing tap water.

The present invention also relates to a method for suppression of lumpy fat, which uses electrolytically reduced water for bathing purposes, thereby suppressing the growth of lumpy fat (cutaneous lumpy fat)

### BACKGROUND ART

Among alkali ion water conditioning instruments or conditioners for producing alkali ion water (electrolytically reduced water) and acid ion water from tap water are those which have an ion exchange membrane interposed between an positive electrode and a negative electrode, and utilize an electrolytic action on water to separate water, forming alkali ion water and acid ion water. Such alkali ion water conditioners include those of the type fixed to the faucet (cock) of a water supply, and those of the type disposed halfway through a water pipe, so-called built-in types.

In the alkali ion water conditioner of the built-in type, for example, a user turns on the faucet, whereby tap water is flowed from the water main, and supplied to an electrolytic cell, where alkaline water and acidic water are produced. The alkaline water produced in the electrolytic cell is delivered through a delivery pipe, while the acidic water is discharged through an acidic water discharge pipe. The user turns off the faucet to stop water flow from the water main, thereby stopping the delivery of the alkaline water (see, for example, Patent Document 1).

Because of a decline in metabolism, for example, fat in the body is riot sufficiently metabolized, but builds up, as a lump, around the fat cells. The presence of this so-called lumpy fat has attracted widespread attention, and increased consciousness of health has aroused a demand for the suppression of lumpy fat. Generally, supplements are administered for suppressing lumpy fat, but side effects or adverse reactions caused by their administration actually pose problems. Thus, wider attention begins to be paid to bathing in alkali ion water to activate the skin, thereby suppressing lumpy fat. As the alkali ion water for bathing, it is common practice to use the one from an alkali ion water conditioner which produces alkali ion water and acid ion water from tap water.

It has been known qualitatively to activate the skin with alkali ion water (electrolytically reduced water), thereby suppressing lumpy fat. However, it remains unclear what serves as a measure of the reducing power of electrolytically reduced water capable of suppressing subcutaneous lumpy fat to prevent its oxidative deterioration.

Patent Document 1: JP-A-10-192858

### DISCLOSURE OF THE INVENTION

### Problems to be Solved by the Invention

The present invention has been accomplished in the light of the above-described circumstances. It is an object of the invention to provide electrolytically reduced water which is quantitatively defined in terms of the reducing power capable of activating the skin to prevent oxidative deterioration and suppress subcutaneous lumpy fat (cellulite). It is another object of the invention to provide electrolytically reduced water which is quantitatively defined in terms of the reducing power capable of inhibiting the formation of lumpy fat and burning fat.

The present invention has also been accomplished in the light of the above-described circumstances. It is an object of the invention to provide hot water for bathing, which can activate the skin to prevent oxidative deterioration and suppress subcutaneous lumpy fat (cellulite). It is another object of the invention to provide hot water for bathing, which can inhibit the formation of lumpy fat and burn fat.

The present invention has also been accomplished in the light of the above-described circumstances. It is an object of the invention to provide a method for suppression of lumpy fat, which can activate the skin to prevent oxidative deterioration and suppress subcutaneous lumpy fat (cellulite). It is another object of the invention to provide a method for suppression of lumpy fat, which can inhibit the formation of lumpy fat and burn fat.

### Means for Solving the Problems

Electrolytically reduced water of the present invention according to claim 1 for attaining the above object is characterized by having a dissolved hydrogen concentration of 0.20 ppm or higher.

Because of this feature, the oxidative deterioration of fat in the cutis or skin can be prevented through the elimination of active oxygen due to the characteristics of hydrogen. Moreover, the formation of lumpy fat can be inhibited, and fat can be burned, owing to the characteristics of active hydrogen.

Electrolytically reduced water of the present invention according to claim 2 for attaining the above object is characterized by having an oxidation-reduction potential of −150 mV to −500 mV.

Because of this feature, the oxidative deterioration of fat in the skin can be prevented through the elimination of active oxygen due to the oxidation-reduction potential characteristics with high reducing action. Moreover, the formation of lumpy fat can be inhibited, and fat can be burned, owing to the oxidation-reduction potential characteristics with high reducing action.

Electrolytically reduced water of the present invention according to claim 3 for attaining the above object is characterized by having a dissolved hydrogen concentration of 0.20 ppm or higher and an oxidation-reduction potential of −150 mV to −500 mV.

Because of this feature, the oxidative deterioration of fat in the skin can be prevented through the elimination of active oxygen due to the characteristics of hydrogen and the oxidation-reduction potential characteristics with high reducing action. Moreover, the formation of lumpy fat can be inhibited, and fat can be burned, owing to the characteristics of active hydrogen and the oxidation-reduction potential characteristics with high reducing action.

Electrolytically reduced water of the present invention according to claim 4 for attaining the above object is characterized by having a dissolved hydrogen concentration of 0.20 ppm or higher, an oxidation-reduction potential of −150 mV to −500 mV, and hydrogen ion exponent of pH 6.5 to pH 10.5.

Because of this feature, the oxidative deterioration of fat in the skin can be prevented through the elimination of active oxygen due to the characteristics of hydrogen, the oxidation-reduction potential characteristics with high reducing action, and alkaline characteristics. Moreover, the formation of lumpy fat can be inhibited, and fat can be burned, owing to the characteristics of active hydrogen, the oxidation-reduction potential characteristics with high reducing action, and alkaline characteristics.

Electrolytically reduced water of the present invention according to claim 5 is the electrolytically reduced water according to any one of claims 1, 3 and 4, characterized in that the dissolved hydrogen concentration is 1.00 ppm or lower.

Because of this feature, in which the dissolved hydrogen concentration is 1.00 ppm or lower, hydrogen resides in a microbubble state within the liquid, so that an indicator of hydrogen can be rendered more accurate.

Electrolytically reduced water of the present invention according to claim 6 is the electrolytically reduced water according to claim 4, characterized in that the hydrogen ion exponent is pH 9.5 to pH 10.0.

Because of this feature, the hydrogen ion concentration can be defined more accurately.

The above-mentioned electrolytically reduced water has temperature characteristics which can exceed the dissolved hydrogen concentration of 0.20 ppm or higher and the oxidation-reduction potential of −150 mV to −500 mV, even when it is at a temperature of 39° C. to 44° C. which essentially poses difficulty in maintaining the dissolved hydrogen concentration and the oxidation-reduction potential.

Electrolytically reduced water of the present invention according to claim 7 is the electrolytically reduced water according to any one of claims 1 to 6, further comprising a hydrogen absorbing holder for absorbing dissolved hydrogen.

Because of this feature, in which the hydrogen absorbing holder absorbs dissolved hydrogen, it becomes possible to retain dissolved hydrogen at a high concentration and for a long time. Thus, the formation of lumpy fat (cutaneous lumpy fat) can be inhibited for a long time, and fat can be burned, owing to the characteristics of active hydrogen.

Electrolytically reduced water of the present invention according to claim 8 is the electrolytically reduced water according to claim 7, characterized in that the hydrogen absorbing holder comprises a natural or naturally incorporated water-soluble polymer.

Because of this feature, dissolved hydrogen can be absorbed using the natural or naturally incorporated water-soluble polymer.

Electrolytically reduced water of the present invention according to claim 9 is the electrolytically reduced water according to claim 8, characterized in that the natural or naturally incorporated water-soluble polymer is carboxymethylcellulose sodium (CMC sodium).

As the natural or naturally incorporated water-soluble polymer, there can be applied one polymer or a plurality of polymers selected from the group consisting of CMC sodium, carboxymethyl cellulose (CM cellulose) fucoidan, guar gum, and dextranic acid.

The CM cellulose preferably has a degree of substitution, as a carboxymethylation rate, of 0.2 to 0.9, preferably 0.3 to 0.5. The CM cellulose preferably has a degree of polymerization, as the molecular weight of the cellulose, of 500 to 7,000, preferably 1,000 to 3,000. The CM cellulose is preferably the one containing a sodium salt, a potassium salt, aluminum or the like as an incorporated metal.

Electrolytically reduced water of the present invention according to claim 10 is the electrolytically reduced water according to claim 7, characterized in that the hydrogen absorbing holder comprises a water-soluble synthetic polymer.

As the water-soluble synthetic polymer, there can be applied one or a plurality of polymers selected from the group consisting of polyethylene glycol (PEG) and polyvinyl pyrrolidone (PVP).

Electrolytically reduced water of the present invention according to claim 11 is the electrolytically reduced water according to claim 7, characterized in that the hydrogen absorbing holder comprises an antioxidant,

As the antioxidant, there can be applied one or a plurality of antioxidants selected from the group consisting of hesperidin, ascorbic acid, and catechin.

In addition to the natural water-soluble polymer, the water-soluble synthetic polymer, and the antioxidant, there can be applied natural fats and oils, including Citrus junos oil.

Hot water for bathing of the present invention according to claim 12 for attaining the above object is characterized by using the electrolytically reduced water according to any one of claims 1 to 11, and being applied to the suppression of the oxidative deterioration of fat in the skin.

Because of this feature, hot water for bathing becomes available which can prevent the oxidative deterioration of fat in skin. Moreover, hot water for bathing can be obtained which can inhibit the formation of lumpy fat, and can burn fat.

A method for suppression of lumpy fat of the present invention according to claim 13 for attaining the above object is characterized by using the electrolytically reduced water according to any one of claims 1 to 11 for bathing, and suppressing the oxidative deterioration of fat in the skin to suppress the growth of lumpy fat, inhibiting the formation of lumpy fat, and burning fat.

Because of this feature, the method for suppression of lumpy fat becomes available which can prevent the oxidative deterioration of fat in skin. Moreover, the method for suppression of lumpy fat can be obtained which can inhibit the formation of lumpy fat, and can burn fat.

### Effects of the Invention

The electrolytically reduced water of the present invention can be obtained as electrolytically reduced water which is quantitatively defined in terms of the reducing power capable of activating the skin to prevent oxidative deterioration, performing an eliminating action on active oxygen, and suppressing subcutaneous lumpy fat. There can also be provided the electrolytically reduced water which is quantitatively defined in terms of the reducing power capable of inhibiting the formation of lumpy fat and burning fat.

The hot water for bathing of the present invention can be obtained as hot water for bathing which can prevent the oxidative deterioration of fat in the skin. There can also be provided the hot: water for bathing which can inhibit the formation of lumpy fat and burn fat.

The method for suppression of lumpy fat of the present invention can be obtained as a method for suppression of lumpy fat which can prevent the oxidative deterioration of fat in the skin. There can also be provided the method for suppression of lumpy fat which can inhibit the formation of lumpy fat and burn fat.

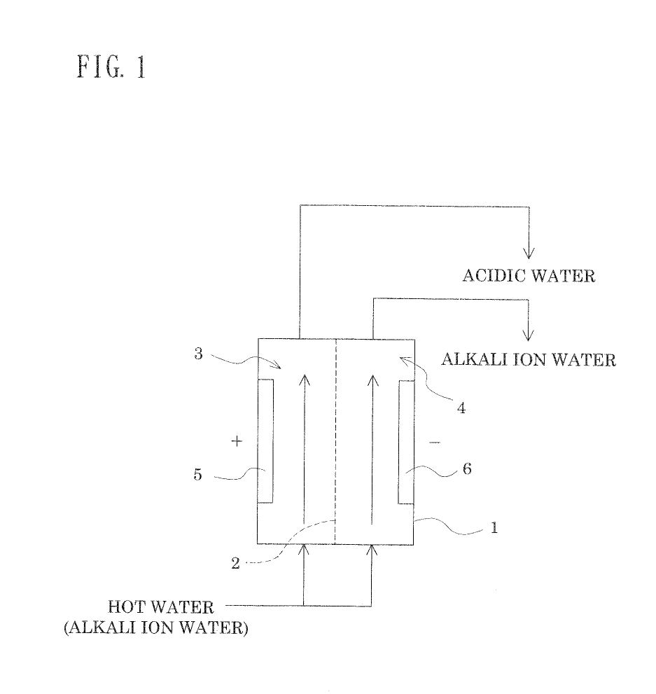

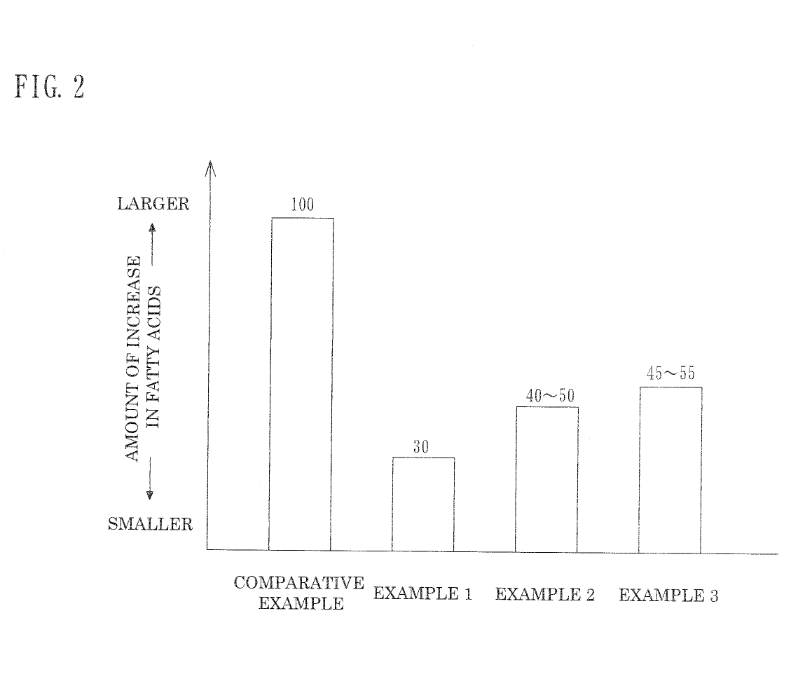

### BRIEF DESCRIPTION OF THE DRAWINGS

[FIG. 1] is a schematic configurational drawing of an apparatus for producing alkali ion water.

[FIG. 2] is a graph showing increases in cellulite precursors.

### DESCRIPTION OF THE NUMERALS AND SYMBOLS

1 Electrolytic cell

2 Ion-exchange membrane

3 Positive-side passage

4 Negative-side passage

### BEST MODE FOR CARRYING OUT THE INVENTION

The electrolytically reduced water according to embodiments of the present invention is alkali ion water obtained by ion exchange. The electrolytically reduced water has a dissolved hydrogen concentration of 0.20 ppm or higher, an oxidation-reduction potential (a larger negative value represents a stronger reducing power, suppressing oxidation more potently) of −150 mV to −500 mV, and a hydrogen ion exponent of pH 6.5 to pH 10.5. At a temperature of 41° C., for example, the electrolytically reduced water has a dissolved hydrogen concentration of 0.52 ppm and an oxidation-reduction potential of −150 mV to −500 mV. The hydrogen ion exponent is preferably pH 9.0 or higher, but the present invention may use electrolytically reduced water of pH 6.5 or higher, for example, upon addition of a pH neutralizer.

The electrolytically reduced water has the temperature characteristics which can exceed the dissolved hydrogen concentration of 0.20 ppm or higher and the oxidation-reduction potential of −150 mV to −500 mV, even when it is at a temperature of 39° C. to 44° C. which essentially poses difficulty in maintaining the dissolved hydrogen concentration and the oxidation-reduction potential.

If the dissolved hydrogen concentration is lower than 0.20 ppm, active hydrogen is inadequate. Thus, the electrolytically reduced water is not different from ordinary tap water, and makes it difficult to activate the skin to such a degree that lumpy fat is suppressed. If the oxidation-reduction potential is greater than −150 mV, the reducing power is insufficient, presenting difficulty in preventing the oxidative deterioration of fat in the skin.

Thus, the oxidative deterioration of fat in the skin can be prevented through the elimination of active oxygen due to the characteristics of hydrogen, the oxidation-reduction potential characteristics with high reducing action, and alkaline characteristics. That is, a sufficient concentration of hydrogen is dissolved to obtain a high reducing power, thereby performing an eliminating action on active oxygen to protect against the oxidative deterioration of fat in the skin, inhibit the formation of fat droplets, and suppress lumpy fat. Moreover, owing to the characteristics of hydrogen, the oxidation-reduction potential characteristics with high reducing action, and alkaline characteristics, the skin can be activated to prevent the oxidative deterioration of fat in the skin, inhibit the formation of lumpy fat, and burn fat.

The dissolved hydrogen concentration is preferably 1.00 ppm or lower. It is further preferred for the hydrogen ion exponent to be pH 9.5 to pH 10.0. By so doing, hydrogen resides in a microbubble state within the liquid, so that an indicator of hydrogen can be rendered more accurate. Moreover, the hydrogen ion concentration can be defined more accurately. The temperature of the electrolytically reduced water is preferably 38° C. to 42° C.

If the dissolved hydrogen concentration exceeds 1.00 ppm, hydrogen does not reside in the solution, so that it becomes difficult for hydrogen to be dissolved in a microbubble state.

Hence, there can be obtained electrolytically reduced water which is quantitatively defined in terms of the reducing power capable of activating the skin to suppress subcutaneous lumpy fat, and prevent its oxidative deterioration. There can also be obtained hot water for bathing which prevents the oxidative deterioration of fat in the skin to suppress the formation of lumpy fat. By bathing in hot water for bathing, the oxidative deterioration of fat in the skin can be prevented to suppress the formation of lumpy fat. The skin can also be activated to prevent the oxidative deterioration of fat in the skin, inhibit the formation of lumpy fat, and burn fat.

The hydrogen absorbing holder, which absorbs dissolved hydrogen, can be incorporated into the electrolytically reduced water according to the embodiments of the present invention. By incorporating the hydrogen absorbing holder into the electrolytically reduced water, it becomes possible to retain dissolved hydrogen at a high concentration and for a long time. Because of the characteristics of active hydrogen, the formation of lumpy fat (cutaneous lumpy fat) can be inhibited for a long time, and fat can be burned. As the hydrogen absorbing holder, there can be applied natural or naturally incorporated, water-soluble polymers, water-soluble synthetic polymers, antioxidants, and natural fats and oils.

### Example 1

Electrolytically reduced hot water having a dissolved hydrogen concentration of 0.23 ppm, an oxidation-reduction potential of −204 mV (38.3° C.), and a hydrogen ion exponent of pH 9.5 (32.9° C.) was produced at a bath voltage of 50 V.

### Example 2

Electrolytically reduced hot water having a dissolved hydrogen concentration of 0.33 ppm, an oxidation-reduction potential of −314 mV (40.8° C.), and a hydrogen ion exponent of pH 9.9 (34.1° C.) was produced at a bath voltage of 100 V.

### Example 3

Electrolytically reduced hot water having a dissolved hydrogen concentration of 0.52 ppm, an oxidation-reduction potential of −406 mV (42.5° C.), and a hydrogen ion exponent of pH 10.0 (36.5° C.) was produced at a bath voltage of 150 V.

As the electrolytically reduced hot waters shown in Examples 1 to 3, alkali ion water produced by ion exchange is applied. The outline of an apparatus for producing alkali ion water will be described based on FIG. 1. FIG. 1 shows the schematic configuration of the apparatus for producing alkali ion water.

As shown in the drawing, hot water (e g. 42° C.) is fed to an electrolytic cell 1, and the hot water is ionized inside the electrolytic cell 1 to produce electrolytically reduced hot water (alkali ion water) and acid ion water. An ion-exchange membrane 2 is fixed inside the electrolytic cell 1, and the interior of the electrolytic cell 1 is divided into a positive-side passage 3 and a negative-side passage 4 by the ion-exchange membrane 2. A positive electrode 5 is provided in the positive-side passage 3, while a negative electrode 6 is provided in the negative-side passage 4, and a predetermined voltage is applied between the positive electrode 5 and the negative electrode 6. As a result, acid ion water is produced in the passage 3 by ion exchange, while alkali ion water is produced in the passage 4 by ion exchange.

That is, when hot water is passed between the ion-exchange membrane 2 and the electrode, the hot water is ionized into hydrogen ions H⁺ and hydroxyl ions OH⁻. The hydrogen ions H⁺ gather in one of the spaces via the ion-exchange membrane 2, whereby alkali ion water and acid ion water are produced.

That is, in the passage 4 on the side of the negative electrode 6 among the two spaces, the hydrogen ions H⁺ gather after passing through the ion-exchange membrane 2. The hot water (2H₂O) is conditioned with electrons (2e⁻) into H₂+2OH⁻, whereby alkali ion water having hydrogen dissolved therein is formed. In the passage 3 on the side of the positive electrode 5, on the other hand, the hot water (2H₂O) is conditioned into O₂4H⁺+4e⁻, whereby acid ion water is formed.

By setting the conditions, such as the flow rate and the flow path of hot water, and applied voltage, as appropriate, the alkali ion waters (electrolytically reduced hot waters) of Examples 1 to 3 can be obtained. Aside from the conditions so set, alkali ion water obtained separately, or the alkali ion water obtained upon flowing through the electrolytic cell 1, is flowed again through the negative-side passage 4 of the electrolytic cell 1, whereby the alkali ion waters of Examples 1 to 3 can be obtained. If necessary, moreover, a hydrogen gas is bubbled from the outside through the alkali ion water, whereby the dissolved hydrogen concentration can he brought to a predetermined state.

### Verification Example

Skin tissues incorporating desired fatty acids were cultured with the use of the alkali ion waters of Examples 1 to 3, and the results of the cultures were verified. The experimental procedure consisted of adding the alkali ion waters of Examples 1 to 3 to the skin tissues for 60 minutes, then rinsing the tissues, and allowing them to stand. This procedure was performed at intervals of 24 hours for 72 hours (namely, performed 3 times). In detail, the state where after bathing for 60 minutes, a normal life was led, and a bath was taken 23 hours later was repeated 3 times.

Investigations were conducted into the status of formation of cellulite precursors (fat droplets) in the skin tissues, namely, the manner of increases of fatty acids present in the skin tissues (i.e., the amounts of oxidized fat), upon addition of the alkali ion waters of Examples 1 to 3, and upon addition of ordinary hot water (Comparative Example). The results are shown in FIG. 2. FIG. 2 shows a graph revealing increases in cellulite precursors when the alkali ion waters of Examples 1 to 3 and ordinary hot water were added.

As shown in the drawing, when ordinary hot water was added, and fatty acids were incubated (Comparative Example), the amount of an increase in the fatty acids was taken as 100. In this case, the amount of an increase in the fatty acids when incubated upon addition of the electrolytically reduced hot water of Example 1 was of the order of 30, confirming that the increase in the fatty acids was about a third. The amount of an increase in the fatty acids when incubated upon addition of the electrolytically reduced hot water of Example 2 was of the order of 40 to 50, and the amount of an increase in the fatty acids when incubated upon addition of the electrolytically reduced hot water of Example 3 was of the order of 45 to 55. These findings confirmed the increases in the fatty acids of both examples to be about a half.

The addition of the alkali ion waters of Examples 1 to 3 for 30 minutes to the skin tissues was also confirmed to produce effects showing a similar tendency.

Accordingly, the electrolytically reduced hot waters of Examples 1 to 3 were confirmed to suppress the oxidative deterioration of fat in the skin through the elimination of active oxygen due to the characteristics of hydrogen, the oxidation-reduction potential characteristics with high reducing action, and alkaline characteristics. That is, a sufficient concentration of hydrogen is dissolved to obtain a high reducing power, thereby performing an eliminating action on active oxygen, thus protecting against the oxidative deterioration of fat in the skin, inhibiting the formation of fat droplets, and suppressing lumpy fat. Since the suppression of lumpy fat has been confirmed, the formation of lumpy fat can be inhibited, and fat can be burned.

Fatty acids are susceptible to oxidative deterioration, and when subjected to oxidative deterioration, they are solidified and minimally undergo metabolic decomposition called fat combustion, further inducing apoptosis. It is the reducing power that prevents this oxidative deterioration. The electrolytically reduced hot waters of Examples 1 to 3 have a strong reducing power as evidenced by an oxidation-reduction potential of −150 mV to −500 mV. As noted here, a suppressive effect on lumpy fat (curtailment of the amount of an increase in fatty acids) appeared 48 hours after the addition of the electrolytically reduced water for 60 minutes daily. Thus, the suppressive effect on lumpy fat was fully verified. The electrolytically reduced water has dissolved hydrogen eliminate active oxygen generated in the human body, and thus can protect against the oxidative deterioration of fat in the skin, accelerate its metabolic decomposition, and suppress the formation of lumpy fat.

A hydrogen absorbing holder can be incorporated into the alkali ion waters (electrolytically reduced waters) shown in Examples 1 to 3. By incorporating the hydrogen absorbing holder, hydrogen in the alkali ion water is absorbed in the hydrogen absorbing holder to suppress the transpiration of dissolved hydrogen, thereby keeping the dissolved state for a long time. An explanation will be offered below for changes over time in the amount of dissolved hydrogen when carboxymethylcellulose sodium (CMC sodium), a natural water-soluble polymer, was incorporated as the hydrogen absorbing holder.

CMC sodium (1666 ppm) was charged into 30 ml of alkali ion water as a sample, and the mixture was stirred at 1200 rpm. Then, the amount of dissolved hydrogen was measured. When the agent absorbing dissolved hydrogen was not added, the amount of dissolved hydrogen decreased from about 0.7 ppm to about 0.1 ppm in 15 minutes. When the CMC sodium was added, it was confirmed that the amount of dissolved hydrogen decreased only slightly, from about 0.7 ppm to about 0.3 ppm, in 15 minutes. That is, even after a lapse of 15 minutes, the presence of dissolved hydrogen in an approximately 3-fold amount was confirmed. On this occasion, the alkali ion water used had a hydrogen ion exponent of pH 6.5 to pH 10.5, which was confirmed to cause no changes in the effect.

By incorporating the hydrogen absorbing holder, hydrogen which transpires into the atmosphere in a short time is once taken up into the hydrogen absorbing holder until a concentration difference equilibrium is reached. For example, whenever hydrogen within water for bathing is consumed for neutralization, etc. of active oxygen to disturb the concentration difference equilibrium, hydrogen is released from the hydrogen absorbing holder. Thus, the amount of hydrogen dissolved during bathing can be retained at a high level for a long time.

As the natural or naturally incorporated water-soluble polymer serving as the hydrogen absorbing holder, there can be applied one polymer or a plurality of polymers selected from the group consisting of carboxymethyl cellulose (CM cellulose), fucoidan, guar gum, and dextranic acid in addition to CMC sodium. When CM cellulose, fucoidan or guar gum was applied, the amount of dissolved hydrogen, which was about 2 to 3 times that without the incorporation of the hydrogen absorbing holder, was noted after a lapse of 15 minutes. Thus, the application of the natural water-soluble polymer is found to suppress the transpiration of dissolved hydrogen, retaining the dissolved state for a long time.

The CM cellulose preferably has a degree of substitution, as a carboxymethylation rate, of 0.2 to 0.9, preferably 0.3 to 0.5. The CM cellulose preferably has a degree of polymerization, as the molecular weight of the cellulose, of 500 to 7,000, preferably 1,000 to 3,000. The CM cellulose is preferably the one containing a sodium salt, a potassium salt, aluminum or the like as an incorporated metal.

As the hydrogen absorbing holder, there can be applied a water-soluble synthetic polymer comprising one polymer or a plurality of polymers selected from the group consisting of polyethylene glycol (PEG) and polyvinyl pyrrolidone (PVP). As the hydrogen absorbing holder, there can also be applied an antioxidant comprising one or a plurality of antioxidants selected from the group consisting of hesperidin, ascorbic acid, and catechin. Further, natural fats and oils, such as Citrus junos oil, can be applied as the hydrogen absorbing holder.

The use of the electrolytically reduced hot waters of Examples 1 to 3 as hot water for bathing makes it possible to activate the skin by bathing, thereby suppressing the oxidative deterioration of fat in the skin and restraining the growth of lumpy fat.

The hot water for bathing can not only activate the skin to prevent the oxidative deterioration of fat in the skin, but also inhibit the formation of lumpy fat, and burn fat.

Also, the incorporation of CMC sodium as the hydrogen absorbing holder enables the amount of dissolved hydrogen to persist at a high level for a long time, making it possible to inhibit the formation of lumpy fat (cutaneous lumpy fat) and enhance the combustion of fat owing to the characteristics of active hydrogen during bathing.

### INDUSTRIAL APPLICABILITY

The present invention can be used in the field of industry of electrolytically reduced water obtained by ionizing tap water supplied from waterworks.

The present invention can also be used in the field of industry of hot water for bathing, which uses electrolytically reduced water obtained by ionizing tap water.

The present invention can also be used in the field of industry of a method for suppression of lumpy fat, which uses electrolytically reduced water for bathing, thereby suppressing the growth of lumpy fat.

1-13. (canceled)

14. Electrolytically reduced water, characterized by
having a dissolved hydrogen concentration of 0.20 ppm or higher.

15. Electrolytically reduced water, characterized by
having an oxidation-reduction potential of −150 mV to −500 mV.

16. Electrolytically reduced water, characterized by
having a dissolved hydrogen concentration of 0.20 ppm or higher and an oxidation-reduction potential of −150 mV to −500 mV.

17. Electrolytically reduced water, characterized by
having a dissolved hydrogen concentration of 0.20 ppm or higher, an oxidation-reduction potential of −150 mV to −500 mV, and a hydrogen ion exponent of pH 6.5 to pH 10.5.

18. The electrolytically reduced water according to claim 17, characterized in that
the dissolved hydrogen concentration is 1.00 ppm or lower.

19. The electrolytically reduced water according to claim 17, characterized in that the hydrogen ion exponent is pH 9.5 to pH 10.0.

20. The electrolytically reduced water according to claim 19, further comprising
a hydrogen absorbing holder for absorbing dissolved hydrogen.

21. The electrolytically reduced water according to claim 20, characterized in that
the hydrogen absorbing holder comprises a natural or naturally incorporated water-soluble polymer.

22. The electrolytically reduced water according to claim 21, characterized in that
the natural or naturally incorporated water-soluble polymer is carboxymethylcellulose sodium (CMC sodium).

23. The electrolytically reduced water according to claim 20, characterized in that
the hydrogen absorbing holder comprises a water-soluble synthetic polymer.

24. The electrolytically reduced water according to claim 20, characterized in that
the hydrogen absorbing holder comprises an antioxidant.

25. Hot water for bathing, characterized by
using the electrolytically reduced water according to claim 17, and being applied to suppression of oxidative deterioration of fat in skin.

26. A method for suppression of lumpy fat, characterized by
using the electrolytically reduced water according claim 17 for bathing, and
suppressing oxidative deterioration of fat in skin to suppress growth of lumpy fat, inhibiting formation of lumpy fat, and burning fat.
